(19) United States
(12) Patent Application Publication
(10) Pub. No.: US 2006/0147925 A1
(43) Pub. Date: Jul. 6, 2006

(54) Method of detection

(76) Inventors: Alexander Alan Morley, Glenelg (AU); Scott Andrew Grist, Currency Creek (AU)

Correspondence Address: SCULLY, SCOTT, MURPHY & PRESSER, 400 GARDEN CITY PLAZA, SUITE 300, GARDEN CITY, NY, 11530, US

(21) Appl. No.: 10/534,846

(22) PCT Filed: Nov. 13, 2003
(86) PCT No.: PCT/AU03/01497

§ 371 Date: Nov. 21, 2005

(30) Foreign Application Priority Data
Nov. 13, 2002 (AU) 20022953021
Nov. 14, 2002 (AU) 2002952665

(51) Int. Cl. C12Q 1/68

(52) U.S. Cl. 435/6

(57) ABSTRACT

The present invention relates to a method of detecting a population of cells or microorganisms in a subject and, more particularly, to a method for qualitatively and/or quantitatively detecting a clonal population of cells or microorganisms in a subject. The method of the present invention is useful in a range of applications including, but not limited to, diagnosing a condition characterised by the presence of a clonal population of cells or microorganisms (such as a neoplastic condition), monitoring the progression of such a condition, predicting the likelihood of a subject's relapse from a remissive state to a disease state or for assessing the effectiveness of existing therapeutic drugs and/or new therapeutic agents.

### FIELD OF THE INVENTION

The present invention relates to a method of detecting a population of cells or microorganisms in a subject and, more particularly, to a method for qualitatively and/or quantitatively detecting a clonal population of cells or microorganisms in a subject. The method of the present invention is useful in a range of applications including, but not limited to, diagnosing a condition characterised by the presence of a clonal population of cells or microorganisms (such as a neoplastic condition), monitoring the progression of such a condition, predicting the likelihood of a subject's relapse from a remissive state to a disease state or for assessing the effectiveness of existing therapeutic drugs and/or new therapeutic agents.

### BACKGROUND OF THE INVENTION

The reference to any prior art in this specification is not, and should not be taken as, an acknowledgment or any form of suggestion that that prior art forms part of the common general knowledge in Australia.

A clone is generally understood as a population of cells which has descended from a common precursor cell. Diagnosis and/or detection of the existence of a clonal population of cells or organisms in a subject has generally constituted a relatively problematic procedure. For example, in the diagnosis of some neoplasms (which are clonal) and some non-neoplastic clonal conditions such as myelodysplasia, polycythaemia vera or other myoproliferative syndromes, it can be difficult to determine whether the cellular populations observed are in fact clonal. If present, this property is valuable for the purpose of making a diagnosis. In another example, it can be important to detect a clonal population which constitutes only a minor component within a larger population of cells or organisms. This latter requirement is often important in the detection or monitoring of certain neoplasms, in the detection of enlarged clones generated by the immune system and, in terms of microorganisms, in the identification of drug resistant clones which have arisen within a larger microorganismal population.

Generally, the population within which the clone arises corresponds to a population of cells within a particular tissue or compartment of the body. Nevertheless, despite the fact that sampling such a population of cells effectively narrows the examination to a sub group of cells or organisms, this may nevertheless still present a clinician with problems such as the ability to confirm that a large population of cells which are observed in a disease condition in fact correspond to a clonal population of cells and/or identification of a clonal population within a large background population of non-clonal cells or organisms.

Current methods for detecting and/or quantifying clonal populations, such as malignancies, involves the use of known markers which are shared by all cells of the clone. The marker may be a surface antigen, pattern of several surface antigens or a specific molecular change (eg. a specific mutation). However one of the major drawbacks associated with most currently used techniques, in particular the currently used molecular techniques which are based on probing or amplifying DNA of interest, is that there is a prerequisite for nucleotide sequence information in order to design and synthesise suitably specific probes or primers. This necessarily renders such techniques both complex and expensive. In this regard, current methods do vary in their complexity, ease of performance, sensitivity and applicability. In general there is a direct relationship between complexity and sensitivity and the most sensitive methods are usually very complex and time-consuming. Owing to their complexity, many of the current methods of measuring numbers of clonal cells, such as neoplastic cells, are still only suitable for use as research tools and are not suited for wide-spread clinical use.

Accordingly, there is a need to develop improved methods for qualitatively and/or quantitatively detecting the existence of a clonal population of cells or microorganisms within any biological context (ie. irrespective of the level of non-clonal background cellular or micro-organismal material), which methods are sensitive yet simple to routinely perform.

In work leading up to the present invention, the inventors have developed a simple yet sensitive method for detecting clonal populations of cells or microorganisms in any biological sample. The simplicity and sensitivity of this method stems from the fact that the inventors are not restricted to identifying a clonal population based on the identification of a known and unique nucleotide sequence (such as a sequence possessing a particular mutation) or antigen expressed by the clonal population. Rather, the inventors have developed a method based on the identification and analysis of the identity of a clone derived nucleotide sequence relative to the non-identity of the corresponding nucleotide sequence of non-clone derived genetic material.

Specifically, the method developed by the inventors is based on separating nucleic acid molecules from a specific region, such as a specific genomic region, using a separation method, the separative effect of which is dependent on the nucleic acid sequences of the individual molecules, and analysing the sequence identity vs non-identity of the populations of molecules thus separated. Separated nucleic acid molecules corresponding to such a region, where these regions have been isolated from a heterogeneous population of cells will exhibit a heterogeneous sequence distribution comprising many populations of co-localised molecules. This profile occurs due to the existence of non-identity (specifically, heterogeneity) of the nucleotide sequences of these regions. Separated nucleic acid molecules derived from a sample of cells which is dominated by the presence of an expanded clonal population of cells, however, will show a significantly higher level of one population of co-localised molecules relative either to the levels of other co-localised molecules present in that sample (which other molecules could be derived from genetically divergent cells of non-clonal origin which are nevertheless present in the sample) or to the levels of molecules which would be found in a corresponding sample which does not comprise the expanded clonal population of interest. In a particularly surprising aspect, both leukaemic and non-neoplastic clonal disorders can be identified on this basis.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

One aspect of the present invention is directed to a method of detecting a clonal population of cells in a biological sample, which clonal cells are characterised by a diagnostically distinctive nucleic acid region, said method comprising co-localising the subject nucleic acid regions derived from said sample, which co-localisation is based on nucleotide sequence identity, and qualitatively and/or quantitatively detecting the levels of said co-localised nucleic acid regions wherein a higher level of a co-localised nucleic acid region population relative to background levels is indicative of the presence of a clonal population of cells in said sample.

Another aspect of the present invention is directed to a method of detecting a clonal neoplastic population of cells in a biological sample, which clonal neoplastic cells are characterised by a diagnostically distinctive nucleic acid region, said method comprising co-localising the subject nucleic acid regions derived from said sample, which co-localisation is based on nucleotide sequence identity, and qualitatively and/or quantitatively detecting the levels of said co-localised nucleic acid regions wherein a higher level of a co-localised nucleic acid region population relative to background levels is indicative of the presence of a clonal neoplastic population of cells in said sample.

Yet another aspect of the present invention provides a method of detecting a clonal non-neoplastic population of cells in a biological sample, which clonal non-neoplastic cells are characterised by a diagnostically distinctive nucleic acid region, said method comprising co-localising the subject nucleic acid regions derived from said sample, which co-localisation is based on nucleotide sequence identity, and qualitatively and/or quantitatively detecting the levels of said co-localised nucleic acid regions wherein a higher level of a co-localised nucleic acid region population relative to background levels is indicative of the presence of a clonal non-neoplastic population of cells in said sample.

Still another aspect of the present invention provides a method of detecting a clonal microorganism population in a biological sample, which clonal microorganisms are characterised by a diagnostically distinctive nucleic acid region, said method comprising co-localising the subject nucleic acid regions derived from said sample, which co-localisation is based on nucleotide sequence identity, and qualitatively and/or quantitatively detecting the levels of said co-localised nucleic acid regions wherein a higher level of a co-localised nucleic acid region population relative to background levels is indicative of the presence of a clonal microorganism population in said sample.

In yet still another aspect there is provided a method of detecting a clonal immune cell population in a biological sample, which clonal immune cells are characterised by a diagnostically distinctive nucleic acid region, said method comprising co-localising the subject nucleic acid regions derived from said sample, which co-localisation is based on nucleotide sequence identity, and qualitatively and/or quantitatively detecting the levels of said co-localised nucleic acid regions wherein a higher level of a co-localised nucleic acid region population relative to background levels is indicative of the presence of a clonal immune cell population in said sample.

In still yet another aspect there is provided a method of detecting a clonal population of cells in a biological sample derived from a human, which clonal cells are characterised by a diagnostically distinctive nucleic acid region, said method comprising co-localising the subject nucleic acid regions derived from said sample, which co-localisation is based on nucleotide sequence identity, and qualitatively and/or quantitatively detecting the levels of said co-localised nucleic acid regions wherein a higher level of a co-localised nucleic acid region population relative to background levels is indicative of the presence of a clonal population of cells in said sample.

In a further aspect there is provided a method of detecting a clonal population of cells in a biological sample, which clonal cells are characterised by a diagnostically distinctive DNA region, said method comprising co-localising the subject DNA regions derived from said sample, which co-localisation is based on nucleotide sequence identity, and qualitatively and/or quantitatively detecting the levels of said co-localised nucleic acid regions wherein a higher level of a co-localised nucleic acid region population relative to background levels is indicative of the presence of a clonal population of cells in said sample.

In another further aspect there is provided a method of detecting a clonal population of cells in a biological sample, which clonal cells are characterised by a diagnostically distinctive mitochondrial genome, said method comprising co-localising the subject mitochondrial genome derived from said sample, which co-localisation is based on nucleotide sequence identity, and qualitatively and/or quantitatively detecting the levels of said co-localised genome wherein a higher level of a co-localised genome population relative to background levels is indicative of the presence of a clonal population of cells in said sample.

In still another further aspect the present invention is directed to a method of detecting a non-neoplastic clonal population of cells in a biological sample, which non-neoplastic cells are characterised by a diagnostically distinctive mitochondrial genome, or part thereof, said method comprising co-localising the mitochondrial genome, or part thereof, derived from said sample, which co-localisation is based on nucleotide sequence identity, and qualitatively and/or quantitatively detecting the levels of said co-localised genomes wherein a higher level of a co-localised genome population relative to background levels is indicative of the presence of a non-neoplastic clonal population of cells in said sample.

Yet another aspect of the present invention is provides a method for diagnosing and/or monitoring a clonal population of cells in a mammal, which clonal cells are characterised by a diagnostically distinctive nucleic acid region, said method comprising co-localising the subject nucleic acid regions derived from a biological sample derived from said mammal, which co-localisation is based on nucleotide sequence identity, and qualitatively and/or quantitatively detecting the levels of said co-localised nucleic acid regions wherein a higher level of a co-localised nucleic acid region population relative to background levels is indicative of the presence of a clonal population of cells in said sample.

### BRIEF DESCRIPTION OF THE DRAWINGS

FIG. 1 is a schematic representation of the changes in population of molecules containing the nucleic acid region of interest and separated according to sequence differences, for both a population of cells not containing a clonal population (“non-clonal”) and for a population containing a clonal population of cells.

FIG. 2 is an image of a DGGE result showing separation of molecules derived from the mitochondrial D loop and from either leukaemic or non-leukaemic cells from the same individual. Molecules from the leukaemic cells have a different separation point (lower band) from those containing the germ-line sequence (upper band). Also shown are mixing experiments indicating that the molecules from the leukaemic cells can be detected when they comprise as few as 10% of total molecules.

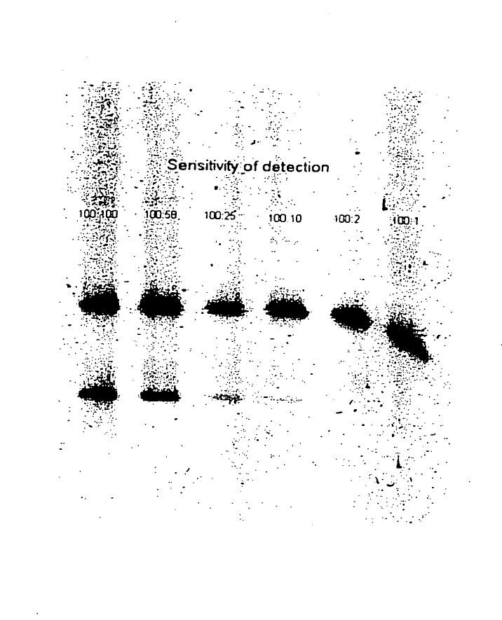

FIG. 3 is an image of the DGGE results for 3 patients with ALL. Patients ALL1 and ALL2 showed a mutated leukemic band which had the same sequence at both diagnosis and relapse. In patient ALL2 the leukemic band is still faintly visible in the remission material. In patient ALL3 the leukemic band was mutated at both diagnosis and relapse but some of the point mutations were different. In this patient both the diagnostic band and the relapse band can also be faintly seen in the remission material.

FIG. 4 is an image of the mixing experiment to determine sensitivity of detection of a minor population by DGGE. Two mitochondrial amplicons derived from 2 normal individuals with different mitochondrial sequences were mixed in various proportions. One was arbitrarily designated the leukemic (L) individual. The L amplicons could be detected down to at least 1%

### DETAILED DESCRIPTION OF THE INVENTION

The present invention is predicated, in part, on the determination that the relative analysis of identity versus non-identity of a specific nucleic acid sequence population provides a simple and efficient means of quickly and accurately identifying the existence of an expanded clonal cellular population in a biological sample of interest. The method of the present invention is facilitated by the availability of highly discriminatory technology which can separate a heterogeneous population of nucleic acid sequences into multiple populations on the basis of differences in actual nucleic acid sequence. The development of a technique which does not rely on knowledge and/or identification of a clone's actual nucleic acid sequence now facilitates the routine analysis, in a high throughput manner, of any biological sample in terms of the presence or absence of one or more clonal populations of cells. In a particularly surprising aspect, this method can be applied to detect both leukaemic and non-neoplastic clonal disorders.

Accordingly, one aspect of the present invention is directed to a method of detecting a clonal population of cells in a biological sample, which clonal cells are characterised by a diagnostically distinctive nucleic acid region, said method comprising co-localising the subject nucleic acid regions derived from said sample, which co-localisation is based on nucleotide sequence identity, and qualitatively and/or quantitatively detecting the levels of said co-localised nucleic acid regions wherein a higher level of a co-localised nucleic acid region population relative to background levels is indicative of the presence of a clonal population of cells in said sample.

Reference to “cells” should be understood as a reference to all forms of cells from any species and to mutants or variants thereof. Without limiting the present invention to any one theory or mode of action, a cell may constitute an organism (in the case of unicellular organisms) or it may be a subunit of a multicellular organism in which individual cells may be more or less specialised (differentiated) for particular functions. All living organisms are composed of one or more cells. The subject cell may form part of the biological sample, which is the subject of testing, in a syngeneic, allogeneic or xenogeneic context. A syngeneic process means that the clonal cell population and the biological sample within which that clonal population exists share the same MHC genotype. This will most likely be the case where one is screening for the existence of a neoplasia in an individual, for example. An “allogeneic” process is where the subject clonal population in fact expresses a different MHC to that of the individual from which the biological sample is harvested. This may occur, for example, where one is screening for the proliferation of a transplanted donor cell population (such as an immunocompetent bone marrow transplant) in the context of a condition such as graft versus host disease. A “xenogeneic” process is where the subject clonal cells are of an entirely different species to that of the subject from which the biological sample is derived. This may occur, for example, where a potentially neoplastic donor population is derived from xenogeneic transplant. Alternatively, to the extent that one is screening for the presence of a clonal microorganism population (such as a bacterial population) in a subject, the presence of the foreign microorganism within a patient is an example of a xenogeneic context. In a related aspect, one may also seek to detect a clonal viral population within a greater viral population. Accordingly, it should be understood that all references to detecting a clonal population of “cells” utilising the method of the present invention should be read as encompassing the detection of a clonal population of virus.

“Variants” of the subject cells include, but are not limited to, cells exhibiting some but not all of the morphological or phenotypic features or functional activities of the cell of which it is a variant. “Mutants” includes, but is not limited to, cells which have been naturally or non-naturally modified such as cells which are genetically modified.

By “clonal” is meant that the subject population of cells has derived from a common cellular origin, in particular, a common ancestor cell. For example, a population of neoplastic cells is derived from a single cell which has undergone transformation at a particular stage of differentiation. In this regard, a neoplastic cell which undergoes further nuclear rearrangement or mutation to produce a genetically distinct population of neoplastic cells is also a “clonal” population of cells, albeit a distinct clonal population of cells. In another example, a T or B lymphocyte which expands in response to an acute or chronic infection or immune stimulation is also a “clonal” population of cells within the definition provided herewith.

Without limiting the present invention to any one theory or mode of action, the clonal cells may or may not comprise an identical genome. In particular, as the ancestral cell and its descendants divide, the original genetic sequence of the ancestral cell is gradually altered in daughter cells, owing to mutation with or without selection. This does not negate the fact, however, that within the cells of a clone there is far more genetic identity than between the various populations of non-clonal cells. Further, and as hereinafter described, the cells which do not belong to the clone of interest may themselves form part of multiple small clone-like populations due to there occurring a degree of cell division in the absence of any significant genetic alteration. However, it should be understood that the method of the present invention is primarily directed to detecting one or more clonal populations which have undergone more significant expansion than is observed in terms of the small “clone-like” populations which are transiently formed as a result of many types of routine cell division. The present invention is also useful for detecting clonal succession. Clonal succession arises where a cell within the clone has undergone a mutation within the clonal sequence, this subclone will then have its own different subclonal sequence and, due to its continued and significant expansion, may be distinguishable from the clonal sequence as it may co-localise at a different point. Clonal succession, ie one subclone after another, is seen in cancer. In yet another example, the clonal population of cells is a clonal microorganism population, such as a drug resistant clone which has arisen within a larger micro-organismal population. Preferably, the subject clonal population of cells is a neoplastic clonal population of cells, a non-neoplastic clonal population of cells, a clonal immune cell population or a clonal microorganism population.

Accordingly, the present invention is preferably directed to a method of detecting a clonal neoplastic population of cells in a biological sample, which clonal neoplastic cells are characterised by a diagnostically distinctive nucleic acid region, said method comprising co-localising the subject nucleic acid regions derived from said sample, which co-localisation is based on nucleotide sequence identity, and qualitatively and/or quantitatively detecting the levels of said co-localised nucleic acid regions wherein a higher level of a co-localised nucleic acid region population relative to background levels is indicative of the presence of a clonal neoplastic population of cells in said sample.

Preferably, said neoplastic population of cells corresponds to a leukaemia, lymphoma or myeloma and most preferably, a leukaemia.

In another preferred embodiment there is provided a method of detecting a clonal non-neoplastic population of cells in a biological sample, which clonal non-neoplastic cells are characterised by a diagnostically distinctive nucleic acid region, said method comprising co-localising the subject nucleic acid regions derived from said sample, which co-localisation is based on nucleotide sequence identity, and qualitatively and/or quantitatively detecting the levels of said co-localised nucleic acid regions wherein a higher level of a co-localised nucleic acid region population relative to background levels is indicative of the presence of a clonal non-neoplastic population of cells in said sample.

Preferably, said non-neoplastic population of cells corresponds to a myelodysplasia, polycythaemia vera or a myeloproliferative syndrome.

In yet another preferred embodiment there is provided a method of detecting a clonal microorganism population in a biological sample, which clonal microorganisms are characterised by a diagnostically distinctive nucleic acid region, said method comprising co-localising the subject nucleic acid regions derived from said sample, which co-localisation is based on nucleotide sequence identity, and qualitatively and/or quantitatively detecting the levels of said co-localised nucleic acid regions wherein a higher level of a co-localised nucleic acid region population relative to background levels is indicative of the presence of a clonal microorganism population in said sample.

In still another preferred embodiment there is provided a method of detecting a clonal immune cell population in a biological sample, which clonal immune cells are characterised by a diagnostically distinctive nucleic acid region, said method comprising co-localising the subject nucleic acid regions derived from said sample, which co-localisation is based on nucleotide sequence identity, and qualitatively and/or quantitatively detecting the levels of said co-localised nucleic acid regions wherein a higher level of a co-localised nucleic acid region population relative to background levels is indicative of the presence of a clonal immune cell population in said sample.

Reference to a “neoplastic cell” should be understood as a reference to a cell exhibiting abnormal “growth”. In this regard, reference to a “non-neoplastic cell” should be understood as a reference to a population of cells which, while they may show some disturbance of growth, do not show the sustained abnormal growth characteristic of neoplasia. The term “growth” should be understood in its broadest sense and includes reference to proliferation. In this regard, an example of abnormal cell growth is the uncontrolled proliferation of a cell. The uncontrolled proliferation of a lymphoid cell may lead to a population of cells which take the form of either a solid tumour or a single cell suspension (such as is observed, for example, in the blood of a leukemic patient). A neoplastic cell may be a benign cell or a malignant cell. In a preferred embodiment, the neoplastic cell is a malignant cell. In this regard, reference to a “neoplastic condition” is a reference to the existence of neoplastic cells in the subject mammal.

Reference to “immune cell” should be understood as a reference to any cell which is directly or indirectly involved in the initiation and/or progression of a specific or non-specific immune response. Preferably, the subject cell is a cell which is involved in the specific immune response and, most preferably, a T cell or a B cell.

Reference to a “biological sample” should be understood as a reference to any sample which is derived from an organism. In this regard, the biological sample may be derivable from any human or non-human organism. Non-human organisms contemplated by the present invention include primates, livestock animals (eg. sheep, pigs, cows, horses, donkeys), laboratory test animals (eg. mice, hamsters, rabbits, rats, guinea pigs), domestic companion animals (eg. dogs, cats), birds (eg. chicken, geese, ducks and other poultry birds, game birds, emus, ostriches), captive wild or tamed animals (eg. foxes, kangaroos, dingoes), reptiles, fish or prokaryotic organisms. Non-human organisms also include plant sources such as rice, wheat, maize, barley or canola. In terms of plant organisms, the method of the present invention is particularly useful, for example, for identifying the colonisation of a plant by either a desirable or undesirable microorganism which has proliferated as a clonal population. For example, one may seek to screen crops for the presence of unique populations of endophytic actinomycetes. Other examples of non-human organisms include bacteria, viruses, parasites, fungi and algae.

It should be understood that the biological sample may be any sample of material derived from the organism. This includes reference to both samples which are naturally present in the organism, such as tissue and body fluids in a mammal (for example biopsy specimens such as lymphoid specimens, blood, lymph fluid, faeces or bronchial secretions) and samples which are introduced into the body of the organism and subsequently removed, such as, for example, the saline solution extracted from the lung following a lung lavage or from the colon following an enema. To the extent that the subject biological sample is a plant organism, the biological sample includes reference to propagation material thereof.

The biological sample which is tested according to the method of the present invention may be tested directly or may require some form of treatment prior to testing. For example, a biopsy sample may require homogenisation prior to testing. Where the sample comprises cellular material, it may be necessary to extract or otherwise expose the nucleic acid material present in the cellular material in order to facilitate analysis of the nucleic acid material in terms of its relative sequence homogeneity. The sample may also require some form of stimulation prior to testing if the test is designed to detect an mRNA marker sequence. In yet another example, the sample may be partially purified or otherwise enriched prior to analysis. For example, to the extent that a biological sample comprises a very diverse cell population, it may be desirable to select out a sub-population of particular interest. For example, to the extent that one is screening for the development of acute myeloid leukaemia, a CD34⁺ or otherwise enriched blood sample provides a means of isolating the myeloid cell component of the blood sample for further analysis. This at least minimises the number of cell types which are analysed by eliminating non-myeloid cells. In another example, it may be desirable to amplify the marker nucleic acid population prior to testing, where specific primers are available, or to amplify the nucleic acid population of the test sample as a whole utilising universal primers, for the purpose of providing a large starting population of nucleic acid molecules. Accordingly, the material analysed in accordance with the method of the present invention may be nucleic acids extracted directly form a biological sample, or could be an artificially created molecules which is a replica of come or all of the population of naturally occurring molecules in the sample. In the latter case, the techniques used to prepare the replicas would preserve information about the relative amounts of each species of molecule present in the original sample. Such techniques, which could include amplification, would be familiar to those of skill in the art.

The choice of what type of sample is most suitable for testing in accordance with the method disclosed herein will be dependent on the nature of the condition which is being monitored. For example, if a neoplastic condition is a leukaemia, a blood sample, lymph fluid sample or bone marrow aspirate would likely provide a suitable testing sample. Where the neoplastic condition is a lymphoma, a lymph node biopsy or a blood or marrow sample would likely provide a suitable source of tissue for testing. Consideration would also be required as to whether one is monitoring the original source of the neoplastic cells or whether the presence of metastases or other forms of spreading of the neoplasia from the point of origin is to be monitored. In this regard, it may be desirable to harvest and test a number of different samples from any one organism. Where the non-neoplastic condition is a myelodysplasia, polycythaemia vera or other myeloproliferative condition, the sample may be of blood or marrow, and, if blood, the cells of myeloid origin may be isolated by positive or negative selection. In another example, to the extent that one may be screening for the normal expansion of a lymphocyte clone, one would preferentially harvest a biological sample from a secondary lymphoid organ or, if the immune response has advanced such that an expanded clonal population has been released into the circulation, one may take a sample of blood or lymph fluid. In still another example, where one is screening for the existence of a clonal population of microorganisms, it would be generally expected that the harvesting of a sample in or around the site of infection of the microorganism (to the extent that the infection is localised as opposed to systemic) would provide a suitable biological sample for testing.

Preferably, the subject biological sample is a human biological sample.

Accordingly, there is preferably provided a method of detecting a clonal population of cells in a biological sample derived from a human, which clonal cells are characterised by a diagnostically distinctive nucleic acid region, said method comprising co-localising the subject nucleic acid regions derived from said sample, which co-localisation is based on nucleotide sequence identity, and qualitatively and/or quantitatively detecting the levels of said co-localised nucleic acid regions wherein a higher level of a co-localised nucleic acid region population relative to background levels is indicative of the presence of a clonal population of cells in said sample.

Preferably, said clonal population of cells is a neoplastic clonal population of cells, a non-neoplastic clonal population of cells, a clonal immune population or a clonal microorganism population.

More preferably, said neoplastic population of cells is a leukaemia, lymphoma, or myeloma and said non-neoplastic population of cells is a myelodysplasia, polycythaemia vera or other myeloproliferative disorder. Most preferably, said neoplastic population is a leukaemia.

The present invention is predicated on the subject clonal population being characterised by a diagnostically distinctive nucleic acid region. Reference to “characterised by” is intended to indicate that the subject cells exhibit the defined characteristic but it is not intended as a limitation in respect of what other characteristics the cell might also exhibit.

Reference to a “nucleic acid region” should be understood as a reference to a part of either the cell's genome or transcriptome. The subject region may be one which is present in all of the cells of an organism or in some cells only. Examples of nucleic acid regions include, but are not limited to, one or more genes or part of a gene. In this regard, the subject region may comprise one or more intron and/or exon regions of a protein encoding gene, or part thereof. Alternatively, the subject gene, or part thereof, may not necessarily encode a protein but may correspond to a non-coding sequence.

The subject nucleic acid region is one which is “diagnostically distinctive”. By this is meant that the nucleotide region is one which is of a length that is feasible to analyse in accordance with the selected means for effecting co-localisation and is sufficiently mutable that it can provide a useful indicator of an expanding clonal population. Preferably, the subject region is 100-500 nucleotides in length where analysis of a single nucleic acid segment occurs. Without limiting the present invention to any one theory or mode of action, an example of a diagnostically distinctive nucleic acid region is one which, in the context of a population a nucleic acid region molecules which have been derived from a normal population of cells, exhibit a substantial proportion of molecules which exhibit a mutated germline sequence. In such a situation, irrespective of whether the diagnostically distinctive nucleic acid region of the clonal population comprises a sequence corresponding to the germline sequence or exhibiting a mutation (which may or may not be shared by a proportion of the non-clonal cells), the relative analysis of these molecules in terms of their co-localised separation points will indicate the existence or not of an expanding population of clonal cells.

As detailed above, and without limiting the present invention in any way, this nucleotide region may comprise the germline sequence or it may comprise a mutated germline sequence, depending on whether or not the ancestral cell of the clone contained a germline or mutated germline sequence In this regard, if one considers a population of nucleic acid molecules derived from cells which do not form part of the clonal population of interest, there will generally occur one dominant population of identical molecules, which will likely represent the germline sequence for that region, together with a number of other bands or a smear, which represent variously mutated sequences. Thus, if desired, the germline sequence can, in fact, be routinely identified since it will correspond to the most prominent population of co-localised molecules in the context of a population of cells derived from a normal control sample. Analysis of the co-localised nucleic acid region populations of a test sample relative to the co-localised nucleic acid region populations of one or more control/standard samples (the results of which control/standard analysis, to the extent that one or more is utilised, forms part of the “background” as hereinafter defined) would enable one to determine, in addition to the existence of a clonal population, whether that clonal population contains the germline sequence, in the context of the region of interest, or a mutated germline sequence and the precise co-localisation point of the subject nucleic acid region of the clonal population. Importantly, this analysis does not require the actual determination of the sequence or sequences involved.

The subject nucleic acid region may be DNA or RNA, such as mRNA. Where the nucleic acid region is a DNA molecule which encodes a proteinaceous molecule, its transcription may be constitutive or it may require that a stimulatory signal be received by the cell in order to induce its transcription and translation. Since the method of the present invention is directed to analysing the subject nucleic acid region per se, where genomic DNA is the subject of detection it is not material whether the region is transcribed or not. However, if the subject method is directed to analysing mRNA, and the protein encoded by said marker is not constitutively produced, it will be necessary to suitably stimulate the subject cell prior to isolating and analysing the subject mRNA. Such stimulation may be performed either in vitro after the biological sample comprising the subject cells has been harvested from the mammal or a stimulatory signal may be administered to the mammal prior to harvesting of the biological sample. Still further, the diagnostically distinctive sequence of the subject nucleotide region may be already present in the subject cell prior to its clonal expansion, either as a mutated or un-mutated germ-line sequence, or it may be a mutation or foreign sequence, such as a virus or virus-specific molecule, which induces the clonal expansion of the cell (such as is observed with virally transformed neoplastic cells). Preferably, said nucleic acid region is a DNA region.

According to this preferred embodiment, there is provided a method of detecting a clonal population of cells in a biological sample, which clonal cells are characterised by a diagnostically distinctive DNA region, said method comprising co-localising the subject DNA regions derived from said sample, which co-localisation is based on nucleotide sequence identity, and qualitatively and/or quantitatively detecting the levels of said co-localised nucleic acid regions wherein a higher level of a co-localised nucleic acid region population relative to background levels is indicative of the presence of a clonal population of cells in said sample.

As detailed hereinbefore, the method of the present invention is predicated on screening for a relative change in the level of a population of co-localised diagnostically distinctive nucleic acid regions. In a particularly surprising aspect, this method can be applied to detect both leukaemic and non-neoplastic clonal populations. In this regard, it is neither practical nor feasible to screen across the entire genome. Accordingly, the method of the present invention is predicated on selecting a region of DNA or RNA (the subject diagnostically distinctive nucleic acid region) which, within the clone of interest maintains absolute or near absolute identity of sequence but which, across the population of cells which do not form part of this clone would exhibit sufficient heterogeneity of sequence such as to render feasible detection of the subject clonal population via nucleic acid co-localisation studies. Such heterogeneity may occur, for example, due to the existence of single point mutations or polymorphic forms of a given gene among the population of individual cells comprising the biological sample as a whole. Accordingly, the expansion of a clonal population from any one of these cells, which inherently means that the members of the clonal population are completely or very substantially genetically identical, provides a convenient basis for identification. As detailed hereinbefore, this objective is achieved by analysing a nucleic acid region which is sufficiently mutable (in the context hereinbefore discussed) Determining an appropriate nucleic acid region for analysis would be a matter of routine procedure which would be well known to those of skill in the art. For example, one may analyse the co-localisation distribution of a selected population of nucleic acid region molecules which have been derived from a normal biological sample. Where a substantial proportion of these molecules are observed to exhibit a heterogeneous range of sequences (ie a range of mutated sequences), there will have been identified a mutable nucleic acid region which can therefore form the subject of analysis in the context of a biological sample of interest (herein referred to as the “test” sample) in accordance with the method of the present invention. It should be understood that one may seek to analyse a single selected region in order to assess its mutability or one may analyse a genomic section by analysing multiple overlapping nucleic acid segments. For example, the mitochondrial genome is approximately 17 kb in length and may be analysed in terms of multiple overlapping segments in order to identify a nucleic acid region which is sufficiently mutable so as to be appropriate for use in the method of the invention. Without limiting the present invention in any way, the analysis, in this way, of one or more selected nucleic acid regions derived from a normal population of cells provides a means of simply and routinely identifying a nucleic acid region suitable for use in the method of the present invention. However, it should nevertheless be understood that this is merely one example of determining a suitable nucleic acid region and does not exclude any other means, either theoretical or practical, for identifying such a region.

Examples of diagnostically distinctive nucleic acid regions which are preferably examined to determine whether or not the expansion of a clonal population is occurring include mitochondrial DNA (such as mitochondrial D loop DNA), microsatellites and other mutable and/or repetitive sequences.

According to this preferred embodiment there is provided a method of detecting a clonal population of cells in a biological sample, which clonal cells are characterised by a diagnostically distinctive mitochondrial genome, said method comprising co-localising the subject mitochondrial genome derived from said sample, which co-localisation is based on nucleotide sequence identity, and qualitatively and/or quantitatively detecting the levels of said co-localised genome wherein a higher level of a co-localised genome population relative to background levels is indicative of the presence of a clonal population of cells in said sample.

Preferably, said mitochondrial genome is the D loop DNA.

Preferably, said biological sample is a human biological sample and said clonal population of cells is a leukaemia, lymphoma, myeloma, myelodysplasia, polycythaemia Vera or other myeloproliferative syndrome.

Most preferably, said clonal population is a leukaemia or a non-neoplastic clonal population.

Accordingly, one can identify an appropriate region for analysis on the basis of its sufficient mutability, thereby providing one with the capacity to identify, in accordance with the method of the invention, an expanding clonal population. The expansion of the clonal population is identified pursuant to a systematic analysis of a selected co-localised population of diagnostically distinctive nucleic acid region molecules relative to the other co-localised populations which are derived from the test sample and/or the range of co-localised populations which are derived from a control or standard sample (preferably a corresponding normal biological sample). In one particularly preferred embodiment, the method of the present invention is performed by means of a suitable gel separation technique. Accordingly, each of the subject co-localised populations appears as a band which localises to a distinct point on the gel. In this type of scenario one is therefore comparing a selected band of the test sample gel with both the other bands which are evident on the test sample gel and/or the range of bands which are evident on one or more control or standard biological sample gels. In this regard, discussion hereinafter in the context of comparing the “bands” of a gel should be understood as a reference to comparing the co-localised diagnostically distinctive nucleic acid region populations which these bands represent (each of these bands comprising a population of molecules exhibiting sequence identity). However, it should not be understood to infer any limitation in relation to the methodology which the person of skill in the art may elect to use in order to achieve co-localisation of nucleic acid molecules based on sequence identity. Examples of the broad range of methods which one might use to achieve this objective are provided hereinafter and although most of these methods are characterised by the visualisation of co-localised nucleic acid populations in the form of a band on a gel, this may not necessarily always be the case.

In accordance with the preferred embodiment described above, determining whether or not a band (ie co-localised population) occurs at a “higher level” (this phrase being hereinafter defined) establishes, respectively, whether or not that band corresponds to a population of molecules which have been derived from a clonal population of cells, that is, whether or not that band indicates the existence of an expanding clonal population of cells in the test sample. Examples of outcomes which may be observed when one analyses a test sample of interest, in accordance with the method of the present invention (in particular, in accordance with a suitable gel separation technique), relative to a corresponding normal sample (as an example of one form of control/standard sample which one may utilise) include, but are not limited to:
(i) The development of one band only on the test sample gel relative to the development of multiple bands or a smear on the control sample gel. This indicates the existence of one dominant population of identical nucleic acid region molecules in the test sample and the absence of other variously mutated molecules which would have been indicated by the presence of other bands (which other bands may have appeared as discrete bands or as a smear) and which would have co-localised to different separation points on the gel due to the differences in sequence of their diagnostically distinctive nucleic acid regions molecules. This result will indicate that all the cells of the test sample form part of an expanded clonal population.
(ii) Where multiple bands develop on the test sample gel, analysis of each of these bands relative to the bands around it and the bands of the control sample gel may reveal one band of the test sample gel which is more intense than the bands around it and more intense than the correspondingly positioned band of the control sample gel. This darker band will correspond to a population of nucleic acid regions which have been derived from an expanding clonal population and is therefore indicative of the existence of such a population.
(iii) Where multiple bands develop on the test sample gel, analysis of each of these bands relative to the bands around it and the bands of the control sample gel may reveal a diminution in number and/or intensity of all the bands in the test sample but one. Although this one band may not appear any more intense than its counterpart on the control sample gel, the decrease in band intensity and/or the heterogeneity of the other bands on the test gel (which can be determined by conducting an analysis of the characteristics of these bands relative to the corresponding control sample) indicates that the apparently “unchanged” band of the test sample in fact corresponds to an expanding clonal population.

Still without limiting the invention in any way, it should be understood that by analysing each of the bands which appear on the test sample gel, one may identify a singly expanding clonal population. However, it should be understood that it is also possible that one may identify two or more expanding clonal populations—although this will likely tend to occur less commonly. Upon identifying the existence of a clonal population, one may seek to optionally perform any one or more additional tests including, but not limited to:
sequencing the nucleic acid region which corresponds to the clonal population
determining whether the nucleic acid region of the clonal population corresponds to a germline sequence or to a mutated germline sequence (means for simply doing this without actually running a sequencing gel have been hereinbefore described)
perform denaturation/reannealing with or without prior addition of a population of nucleic acid region molecules of known origin and followed by separation, in order to obtain further information about one or more populations of co-localised molecules.
perform quantitative studies by methods such as, but not limited to, densitometry or fluorimetry in order to draw conclusions concerning the relative sizes of populations of co-localised molecules

Without limiting the present invention to any one theory or mode of action, as an approximation, all cells derived from the same fertilised zygote, have the same DNA sequence. More precisely, there is a common ancestral germ-line sequence, which is that of the fertilised zygote, but the genetic sequence in individual cells changes during development. In lymphocytes, as a special case for specific cells and specific genes, rearrangement of the immunoglobulin and/or T cell receptor genes occurs during development and is important in generating the immune repertoire. However, as a general situation, all cells are also subject to random mutation, ie. a change in DNA sequence. This mutation may take the form of, but not be limited to, point mutations, deletions, insertions, inversions, duplications, gene amplifications and more gross chromosomal arrangements and may involve any gene or region of DNA. This mutation is sufficiently frequent that it is likely that every cell, somewhere in its genome, bears one or more mutations and, since these mutations are likely to be different from one cell to another, every cell can be regarded as being genetically unique. The genome of all cells will show a common overall pattern, the germ-line pattern, but there will be subtle differences from one cell to another, the number of differences being greater the further apart in development the cells are. Within this overall pattern of random mutation, there will be superimposed mutations which are the consequence of some cell types eg. lymphocytes, being more mutable than others and some regions of the genome likewise being more mutable than other regions.

Accordingly, reference to “identity” should be understood as a reference to identity in respect of the actual nucleotide sequence of the nucleic acid region which is the subject of testing. The selection of a suitable marker region for analysis will fall within the person of skill in the art. For example, to the extent that one is screening for the normal clonal expansion of a lymphoid population subsequently to infection, the marker may be a rearranged genomic variable region of a T cell receptor chain or an immunoglobulin chain. Reference to detecting the “level” of co-localised sequence should be understood as a reference to either qualitatively and/or quantitatively assessing the amount of nucleic acid region molecules exhibiting an identical sequence. At its simplest, assessment by eye of the intensity of the bands which have developed, after staining, on a gel relative to one another or to a control sample may be performed, wherein a darker and/or thicker band is indicative of a higher concentration of localised molecules than a fainter and/or thinner band. More sophisticated analysis can be performed utilising equipment such as a densitometer based on visible light or fluorescence, which can empirically calculate the concentration of nucleic acid sequence co-localised to a given band relative to a standard.

Accordingly, the method of the present invention is predicated on assessment of the levels of co-localised nucleic acid region molecules relative to “background” levels. Reference to “background” should be understood as a reference to the co-localised nucleic acid region populations of the test sample, other than the co-localised population which is the subject of analysis in terms of whether or not it represents a clonal population (ie. all the co-localised bands present on a test sample gel other then the band of interest). Reference to “background also encompasses all the co-localised population of any corresponding standard or control samples. This will naturally include reference to the population which, in the control/standard sample, corresponds to the population which is the subject of analysis in the test sample. In this regard, it should therefore be understood that in the context of a diagnostic test in respect of one biological sample, where two or more bands are obtained on the gel (as would be expected where the clonal population of cells is not the only population of cells present in the sample), the person of skill in the art will likely systematically individually analyse some or all of these bands relative to both the bands around the band under analysis and relative to some or all of the bands of the control/standard gel. Accordingly, the bands which comprise the “background” for any given band which is the subject of analysis will vary slightly. Specifically, the background will always include the bands of the control/standard gel but in terms of the test sample gel, will only include the bands other than the band which is the subject of analysis. Although it is preferable to analyse a band of interest relative to all the band comprising the background, the present invention should be understood to extend to the situation where one analyses a band of interest relative to only part of the background—such as a defined subset of bands. It should be understood that these background results may appear as multiple discrete bands, or as a smear.

In certain disease states the clonal population may, in fact, comprise the only population of cells which is present in the given biological sample. For example, in myelodysplasia, virtually all the cells of the myeloid lineage are clonal. Accordingly, harvesting of an appropriate biological sample for analysis may in fact correspond to the harvesting of a sample which comprises a single population of cells. In such a situation, there would only be a single population of nucleic acid regions co-localised since these regions would all contain the same nucleotide sequence. In this situation, the “background” in fact equates to the absence of co-localised populations of the subject nucleic acid region populations.

It should also be understood that the source of background which is utilised in any given clinical situation may vary. For example, in most situations it will be desirable that the bands of a test sample gel are analysed both internally (ie. relative to one another) and externally (ie. relative to a corresponding normal sample). However, in some situations, such as the situation where one is monitoring the progress of a leukaemic condition, one may seek to analyse the bands of a test sample relative to the other bands of the test sample gel and relative to a previous test sample from that patient and not a normal control. Such a sample should nevertheless be understood to fall within the scope of a “control/standard” sample since it functions as the standard relative to which the test sample is analysed. In a monitoring situation, one will have previously determined the separation point of the diagnostically distinctive nucleic acid region molecules of the clonal population. Accordingly, it may not be necessary to analyse the test sample relative to a normal control since analysis relative to an earlier test result will indicate whether the clonal population is expanding or contracting. It is within the skill of the person of skill in the art to determine the appropriate source of “background” which is required to be assessed in the context of any given clinical situation.

It should be understood that the standard/control sample may be pre-prepared utilising a corresponding biological sample which does not contain the clonal cell population of interest. Alternatively, one may harvest a sample from the same individual. Such results can be maintained on a database and thereby provide a standard against which test samples can be additionally analysed. The former is likely to be of particular use where a given cell type has only just commenced clonal expansion therefore resulting in a relatively low signal level. In this regard, comparison of such a result to a known, normal standard would indicate whether a slight increase in the level of a co-localised nucleic acid region population is in fact normal in such a biological sample or whether that increase is in fact indicative of the expansion of a clonal population.

In general, but without limiting the generality of the points detailed above, the expansion of a clonal population will be associated with a diminution in the size of one or more of the cell populations which do not form part of the clonal population.

It should be understood that the detection of a “higher” level of a co-localised population of molecules is the result of a relative analysis. Specifically, the analysis is made relative to one or more of the “background” parameters detailed herein. For example, one may detect a higher level of a co-localised population of cells by virtue of their being a very significant increase in the level of one co-localised population over the background co-localised populations, which latter populations may in fact appear to be unchanged in terms of their level. Alternatively, and more commonly, the increase in level of the co-localised population of interest may occur together with a decrease in the levels of the background populations. In this scenario, a relatively modest increase in the actual level of the co-localised population of interest may be rendered significantly more obvious by virtue of a simultaneous decrease in the levels of background populations. This is of particular relevance, for example, where the sequence of the nucleic acid region which is the subject of separation is shared by the clonal population of interest and one or more populations of cells which are not related to the subject clonal population. In yet another alternative, the subject “higher” level may be due entirely to a decrease in the levels of populations which fall within the scope of “background” populations.

As detailed hereinbefore the present invention is predicated on the co-localisation of a nucleic acid region population based on relative differences in actual nucleic acid sequence. By separating the population of molecules in this manner, molecules exhibiting sequence identity will localise separately to molecules exhibiting a sequence which differs by as little as one nucleotide. Reference to “co-localisation” should therefore be understood as a reference to any method of analysis which achieves this objective. This includes, for example:

(i) Denaturing Gradient Electrophoresis (DGGE)

As a double stranded DNA fragment is subjected to increasingly denaturing conditions (for instance, increasing concentrations of chemicals such as urea and formamide) the complementary strands dissociate in a domain-like way. The position in the gradient where a domain of a DNA fragment starts to melt, and thus stops migrating, is dependent on the nucleotide sequence. The presence of a mutation affects the stability of the region and therefore changes the conditions which can cause the DNA fragment to melt and stop migrating. Thus the presence of a mutation will alter the migration pattern of otherwise identical fragments. This technique has been demonstrated to analyse fragments of DNA of less than 500 base pairs in length. The gel is usually maintained at a controlled temperature throughout the run.
   (ii) Temperature Gradient Denaturing Electrophoresis
This technique is a variation of DGGE which uses a temperature gradient applied to a gel or capillary, and a constant concentration of denaturant;
   (iii) Constant Denaturing Electrophoresis
This technique uses constant conditions on the borderline of denaturation so that DNA duplexes repeatedly denature and reanneal during migration. The extent of denaturation and reannealing, and thus the rate of migration through the gel or capillary, is dependent on sequence.
   (iv) Single Strand Conformational Electrophoresis
This technique uses conditions of mild denaturation so that single DNA strands partly anneal to themselves and thus adopt conformations dependent on their sequences. The rate of migration of individual sequences through the gel or capillary is dependent on conformation.
   (v) Denaturing High Performance Liquid Chromatography (DHPLC)
The basis upon which DHPLC identifies mutations relates to the detection of heteroduplex formation between mismatched nucleotides in double stranded PCR amplified DNA. During reannealing of wild type and mutant DNA a mixed population of heteroduplexes and homoduplexes is created as a result of sequence variation.
The mixed population can be analysed by HPLC under partially denaturing temperatures, which gives rise to the heteroduplexes being eluted from the column earlier than the homoduplexes because of their reduced melting temperature. Analysis can be performed on the samples to determine where the mutations lie.
   (vi) Microarrays.
Microarrays provide a potentially useful means of screening for variations in sequence of the subject nucleic acid region. For example, microarrays can be established wherein probes hybridising to sequential portions of the sequence of the subject region and to all point mutations thereof are used to determine homogeneity and heterogeneity in the distribution of hybridisation. This provides a method of separating marker molecules based on their similarity with or difference to a given consensus.
   (vii) Mass Spectrometry.

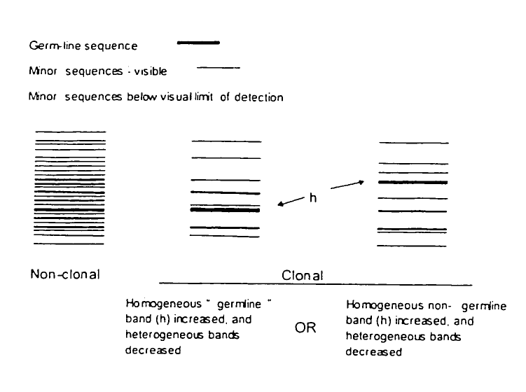

Co-localisation is most conveniently achieved utilising gel or capillary migration technology which facilitates the visualisation of nucleic acid regions which have co-localised by virtue of possessing an identical nucleic acid sequence. For example, the technique of denaturing gel electrophoresis, if performed utilising a biological sample comprising a heterogeneous population of cells, within which one clonal population is expanding, would be expected to result in a series of bands, each one corresponding to a unique sequence of the subject nucleic acid region. Where a significant level of heterogeneity exists in terms of the cells comprising the biological sample, the sample will contain a range of the subject nucleic acid regions which exhibit heterogeneity in terms of their sequences, and this could result in a series of numerous separately localised bands which, to the naked eye, may appear as isolated bands, or as a smear or both (see FIG. 1). Without limiting the present invention in any way, the number of bands visualised may be less than the number of distinct cell types (some of which will exist individually and others of which may themselves form small clonal-like populations due to their division) since some of these cells may not contain a nucleic acid region which exhibits a sequence different to that of the clonal population of interest. In this regard, means of nevertheless utilising the method of the present invention to identify the clonal population of interest have been hereinbefore described. In another example, the number of bands which are visualised may be less than the number of distinct cell types present in the biological sample sue to some populations of co-localised nucleic acid regions being so small as not to be visible. However, to the extent that a clonal population is present in the subject biological sample, the presence of a significantly higher proportion of a nucleic acid region exhibiting a specific nucleic acid sequence, among the other sequences which are present in the sample, will result in the formation of a denser band at the point of separation in the gel or capillary at which this identical population of nucleic acid molecules localise. This will usually, although not necessarily always, also be associated with the disappearance or diminution of a number of the other bands which would be seen in the analysis of a corresponding tissue sample which does not contain the expanding clonal population which is the subject of detection. Preferably, said localisation is performed utilising denaturing gel or capillary electrophoresis.

Preferably, the present invention is directed to a method of detecting a neoplastic clonal population of cells in a biological sample, which neoplastic cells are characterised by a diagnostically distinctive mitochondrial genome, or part thereof, said method comprising co-localising the mitochondrial genome, or part thereof, derived from said sample, which co-localisation is based on nucleotide sequence identity, and qualitatively and/or quantitatively detecting the levels of said co-localised sequences wherein a higher level of a co-localised genome population relative to background levels is indicative of the presence of a neoplastic clonal population of cells in said sample.

More preferably, said neoplastic population of cells is a leukaemia, lymphoma or myeloma and said part thereof of the mitochondrial genome is the D-loop. Most preferably, said neoplastic population of cells is a leukaemia.

In another preferred embodiment, the present invention is directed to a method of detecting a non-neoplastic clonal population of cells in a biological sample, which non-neoplastic cells are characterised by a diagnostically distinctive mitochondrial genome, or part thereof, said method comprising co-localising the mitochondrial genome, or part thereof, derived from said sample, which co-localisation is based on nucleotide sequence identity, and qualitatively and/or quantitatively detecting the levels of said co-localised genomes wherein a higher level of a co-localised genome population relative to background levels is indicative of the presence of a non-neoplastic clonal population of cells in said sample.

Preferably, said non-neoplastic population of cells is a myelodysplasia, polycythaemia vera or other myeloproliferative disorder and said part thereof of the mitochondrial genome is the D-loop.

It should be understood that the results which are obtained may be used directly or may be applied in or converted to any other suitable format. For example, it may be desirable to convert a level of detection which is based on DNA to a level of detection which is based on cells. One method of achieving this is to further calculate mitochondrial genomes per cell.

The method of the present invention provides a simple yet sensitive method of detecting the presence of clonal populations of cells in a subject. The method of the present invention may be used either as a diagnostic tool or as a tool to monitor the progress of a clonal population of cells in terms of detecting the modulation in size of a population of clonal cells or for detecting the instance of clonal evolution of the clonal population of cells.

The method of the present invention is suitable for use in a number of diagnostic situations. These include, but are not necessarily limited to, one or more of
(i). Where the test sample is already known to consist largely or completely of clonal cells. This is the situation in most cases of leukaemia and many cases of lymphoma and myeloma, at diagnosis. Application of the method will both additionally confirm the presence of clonality by demonstrating an increased level of co-localised molecules relative to background and will also determine, by the separation point of the dominant population of co-localised molecules, whether the diagnostically distinctive nucleic acid region is a germline or mutated germline sequence. Knowing the separation point of this population will be of importance for monitoring as in (iii) below.
(ii). Where the test sample may consist largely or completely of clonal cells and it is desired to confirm or exclude the presence of a clonal population for the purpose of diagnosis. This is the situation in most cases of possible myelodysplasia, polycythaemia vera or other myeloproliferative syndromes. Application of the method will confirm or exclude the presence of clonality by demonstrating the presence or absence of an increased level of co-localised molecules relative to background and, if clonality is confirmed, will also determine, by the separation point of the dominant population of co-localised molecules, whether the diagnostically distinctive nucleotide sequence is a germline or mutated germline sequence.
(iii). Where a sample from the patient has previously been studied as in (i) or (ii) above and a later sample has been obtained in order to monitor the progress of the clonal population during or after treatment. In this instance the cell population will consist largely or completely of non-clonal cells but there may be a small population of clonal cells. The principal co-localised molecules will be germline sequences derived largely or completely from non-clonal cells. If the diagnostically distinctive nucleotide region has been previously determined to be a mutated germline sequence then a small band may be detectable at the separation point of these molecules and the level of this population of co-localised molecules relative to background will give an indication of the size of the clonal population. If the diagnostically distinctive nucleotide region has been previously determined to be an un-mutated germline sequence, then the same analysis can be applied but, for a given clone size, the analysis will be less sensitive at detecting the presence and magnitude of the clonal population.
   The same type of comparative analysis can be applied to detect the presence and magnitude of small subclone arising from within the pre-existing clone provided that the diagnostically distinctive nucleotide region of the subclone differs from that of the clone and from the germline sequence.
(iv) Where a previous sample has not been studied and a test sample is obtained in order to determine whether an abnormal or unusually large clonal population is present within a larger population of non-clonal cells. This situation may be involved, for example, in detection of an emerging clone of neoplastic or non-neoplastic cells, of immune cells, or of drug-resistant cells or organisms Application of the method will also require analysis of standards in order to determine the position of the population of co-localised germline sequences and the normal range for the number, sizes and positions of the populations of mutated germline sequences derived from non-clonal cells. This has been hereinbefore described in detail.

Accordingly, another aspect of the present invention is provides a method for diagnosing and/or monitoring a clonal population of cells in a mammal, which clonal cells are characterised by a diagnostically distinctive nucleic acid region, said method comprising co-localising the subject nucleic acid regions derived from a biological sample derived from said mammal, which co-localisation is based on nucleotide sequence identity, and qualitatively and/or quantitatively detecting the levels of said co-localised nucleic acid regions wherein a higher level of a co-localised nucleic acid region population relative to background levels is indicative of the presence of a clonal population of cells in said sample.

Preferably, the clonal population of cells is a neoplastic or non-neoplastic population of cells, a clonal immune cell population or a clonal microorganism population.

Still more preferably, said neoplastic population of cells is a leukaemia, lymphoma or myeloma, said non-neoplastic population of cells is a myelodysplasia, polycythaemia vera or other myeloproliferative disorder and said clonal immune cell population is an activated T cell or B cell antigen-specific population.

Most preferably, said neoplastic population of cells is a leukaemia and said non-neoplastic population of cells is a myelodysplasia or polycythaemia vera.

With respect to this aspect of the present invention, reference to “monitoring” should be understood as a reference to testing the subject for the presence or level of the subject clonal population of cells after initial diagnosis of the existence of said population. “Monitoring” includes reference to conducting both isolated one off tests or a series of tests over a period of days, weeks, months or years. The tests may be conducted for any number of reasons including, but not limited to, predicting the likelihood that a mammal which is in remission will relapse, monitoring the effectiveness of a treatment protocol, checking the status of a patient who is in remission, monitoring the progress of a condition prior to or subsequently to the application of a treatment regime, in order to assist in reaching a decision with respect to suitable treatment or in order to test new forms of treatment. The method of the present invention is therefore useful as both a clinical tool and a research tool.

Yet another aspect of the present invention is directed to a method for diagnosing and/or monitoring a mammalian disease condition characterised by the presence of a clonal population of cells, which clonal cells are characterised by a diagnostically distinctive nucleic acid region, said method comprising co-localising the subject nucleic acid regions derived from a biological sample derived from said mammal, which co-localisation is based on nucleotide sequence identity and qualitatively and/or quantitatively detecting the levels of said co-localised nucleic acid regions wherein a higher level of the co-localised nucleic acid region population relative to background levels is indicative of the presence of a clonal population of cells in said sample.

In one particular aspect, said disease condition is characterised by a neoplastic population of cells and, still more particularly, said disease condition is a leukaemia, lymphoma or myeloma.

In another aspect, said disease condition is characterised by a non-neoplastic population of cells and, still more particularly, said disease condition is a myelodysplasia, polycythaemia vera or other myeloproliferative disorder.

In a most preferred embodiment, said mammal is a human.

Further features of the present invention are more fully described in the following non-limiting Examples.

### EXAMPLE 1

### Protocol for Determination of Sequence Identity or Diversity by DGGE

Mitochondrial DNA is amplified by a PCR process. The amplification protocol used is a two-round PCR, utilising nested primers and performed with a high fidelity or proof reading enzyme, in order to minimise artifactually mutated products that may interfere with the analysis. Primers used in the second round of PCR are engineered to include a GC rich clamp sequence and are carefully chosen to amplify a domain with uniform melting behaviour, as is normally considered good practice for DGGE analysis.

This material is electrophoresed on a polyacrylamide gel with a gradient of denaturants, as per standard DGGE protocols. In terms of normal DGGE protocols however, the gradient of denaturants used for this work would be considered a very “narrow” range, this has the effect of magnifying very small differences in melting behaviour. This gradient is chosen empirically to maximise the separation of all potential sequence variants in the amplicon to be analysed.

Amplicons for analysis are loaded singly in undenatured form, to identify native bands present. Additionally, either the amplicons from the test sample alone or amplicons from the test sample mixed with amplicons from a standard sample are subjected to denaturation followed by a slow renaturation, in order to promote heteroduplex formation between amplicon sub-species that contain sequence differences and thus to make more evident any sequence heterogeneity that is present in the test sample.

After electrophoresis, gels are stained in SYBR green stain and visualised by laser excitation and emission fluorescent detection.

TABLE 1
Results from determination of the DNA sequence of 2 segments
of the mitochondrial D loop in 10 patients with acute myeloblastic
leukaemia at diagnosis and remission. The remission is the control
sequence as it represents predominantly non-leukaemic cells.
Mutations were found in the diagnosis sequence in 4 of the 10
patients. These results indicate that a mutated region of interest
is common in acute myeloid leukaemia. They are not presented
to imply that determination of the DNA sequence is required for
the purpose of the present invention.

| | | | Sequencing | |
|---|---|---|---|---|
| | | | seg d1 | seg d2 |
| | Sample | | nt | nt |
| Disease | ID | Tissue | 16111-16430 | 16411-190 |
| AML | 114/97 | Diag marrow | no marker | no marker |
| | | | found | found |
| | 128/97 | Rem marrow | reference | reference |
| | | | sequence | sequence |
| AML | 89/98 | Diag marrow | no marker | ? |
| | | | found | |
| | 103/98 | Rem marrow | reference | no sequence |
| | | | sequence | |
| AML | 90/98 | Diag marrow | no marker | no marker |
| | | | found | found |
| | 102/98 | Rem marrow | reference | reference |
| | | | sequence | sequence |
| AML | 80/99 | Diag marrow | C-T 16395 | no marker |
| | | | LOH 16230, | found |
| | | | 16278 | |
| | | | LOH 16284, | |
| | | | 16302 | |
| | 101/99 | Rem marrow | reference | reference |
| | | | sequence | sequence |
| AML | 180/98 | Diag marrow | ? | C-T 149 |
| | | | | LOH 16, 73, |
| | | | | 16519 |
| | 13/99 | Rem marrow | no sequence | reference |
| | | | | sequence |
| AML | 35/99 | Diag marrow | 18× point | no marker |
| | | | mutations | found |
| | 55/99 | Rem marrow | reference | reference |
| | | | sequence | sequence |
| AML | aml1 | Diag marrow | no marker | no marker |
| | | | found | found |
| | aml2 | Rem marrow | reference | reference |
| | | | sequence | sequence |
| AML | aml3 | Diag marrow | no marker | T -A/T 8 |
| | | | found | C-C/T |
| | | | | 16519 |
| | aml4 | Rem marrow | reference | reference |
| | | | sequence | sequence |
| AML | aml5 | Diag marrow | no marker | no marker |
| | | | found | found |
| | aml6 | Rem marrow | reference | reference |
| | | | sequence | sequence |
| AML | aml7 | Diag marrow | no marker | no marker |
| | | | found | found |
| | aml8 | Rem marrow | reference | reference |
| | | | sequence | sequence |

TABLE 2
Results from determination of the DNA sequence of 2 segments
of the mitochondrial D loop in 11 patients with acute lymphoblastic
leukaemia at diagnosis and remission. The remission is the control
sequence as it represents predominantly non-leukaemic cells.
One patient (164/165) was not analyzable. Mutations were found
in the diagnosis sequence in 5 of the other 10 patients These
results indicate that a mutated region of interest is common
in acute lymphoblastic leukaemia. They are not presented to imply
that determination of the DNA sequence is required for the purpose
of the present invention.

| | | | |
|---|---|---|---|
| | Sample | | |
| Disease | ID | Tissue | Sequencing |

| | | | | |
|---|---|---|---|---|
| ALL | 171 | Diag | no marker | no marker |
| | | | found | found |
| | 172 | Rem | reference | reference |
| | | | sequence | sequence |
| ALL | 164 | Diag | analysis | no sequence |
| | | | too difficult | |
| | 165 | Rem | heteroplasmic | no sequence |
| | | | ins/del | |
| ALL | 160 | Diag | no marker | no marker |
| | | | found | found |
| | 161 | Rem | reference | reference |
| | | | sequence | sequence |
| ALL | 142 | Diag | C-T 16256, | no marker |
| | | | C-T 16270 | found |
| | | | LOH 16325 | |
| | 143 | Rem | reference | reference |
| | | | sequence | sequence |
| ALL | 274 | Diag | no marker | C-C/T |
| | | | found | 16519 |
| | 275 | Rem | reference | reference |
| | | | sequence | sequence |
| ALL | 178 | Diag | no marker | no marker |
| | | | found | found |
| | 179 | Rem | reference | reference |
| | | | sequence | sequence |
| ALL | 318 | Diag | no marker | no marker |
| | | | found | found |
| | 319 | Rem | reference | reference |
| | | | sequence | sequence |
| ALL | 252 | Diag | T-C 16255, | no marker |
| | | | T-C 16269 | found |
| | | | T-C 16297, | |
| | | | C-T 16326 | |
| | 253 | Rem | reference | reference |
| | | | sequence | sequence |
| ALL | 218 | Diag | 7× point | C-T 16519, |
| | | | mutations | G-A 16526 |
| | | | | LOH 16, 73, |
| | | | | 92, 152 |
| | 219 | Rem | reference | reference |
| | | | sequence | sequence |
| ALL | 827 | Diag | no marker | no marker |
| | | | found | found |
| | 828 | Rem | reference | reference |
| | | | sequence | sequence |
| ALL | 900 | Diag | LOH 16183, | no sequence |
| | | | Del T 16189 | |
| | 903 | Rem | reference | no sequence |
| | | | sequence | |

### EXAMPLE 2

### Use of Denaturing Gradient Gel Electrophoresis in Patients with Acute Myeloid Leukaemia (AML) and Acute Lymphoblastic Leukaemia (ALL)

Methods

There were 22 patients with AML; 11 were males, 11 were females and their ages ranged from 16 to 69. There were 26 patients with ALL; 14 were males, 12 were females, 24 were children with ages ranging from 6 months to 11 years and 3 were adults aged 21, 33 and 69. Fifteen of the ALL patients were selected for study. The material studied was either from cells collected and frozen or from cells spread onto marrow slides. Studies were performed in parallel using material obtained at diagnosis as the source of leukaemic DNA and marrow obtained at the end of induction treatment as the source of constitutional DNA. Only patients who were reported as being in morphological remission at the end of induction were studied. Remission marrow was regarded as an appropriate control source for constitutional DNA, as the leukaemic and non-leukaemic cells had the same or similar tissue of origin and as it would be impossible for any mutations induced by a few weeks of chemotherapy to become sufficiently frequent to be detectable.

For sequencing, the DNA of the D-loop was amplified by the polymerase chain reaction (PCR) in two segments from nucleotides 16111-16430 and 16411-190 and sequenced in both directions on an ABI 373 sequencer. For analysis by denaturing gradient gel electrophoresis (DGGE), the DNA of the D-loop from nucleotide 16071-190 was first amplified as a single segment in a high fidelity first round PCR. (Elongase, New England Biolabs). A second round PCR (Amplitaq Gold, Applied Biosystems) for each of four overlapping segments was then performed using GC clamped primers (nucleotides 16071-16260 5′ Clamp, 16251-16430 5′ Clamp, 16411-100 3′ Clamp, 101-190 3′ Clamp). The amplified material was electrophoresed at 120V for 20 hours through a 10% polyacrylamide gel using a 30-50% urea formamide gradient at a temperature of 60° C. and the separated products analysed using a Molecular Dynamics Fluorimager 595.

For studies to assess the level of detection possible by DGGE, each experiment used mixtures of DNA which had been obtained from two normal individuals, who had been chosen such that the amplified materials denatured at different points in the gradient gel, presumably owing to a sequence difference. Leukaemic DNA mixed with remission DNA from the same individual was not used, as the presence of a minor amount of leukaemic DNA in the remission material might have biased the results.

To convert a level of detection which was based on DNA to a level of detection which was based on cells, mitochondrial genomes/cell were measured by quantitative PCR on a Corbett Rotorgene with detection by fluorescence resonance energy transfer and PCR amplification of bases 1262-1361 of the mitochondrial genome and bases 88946-89014 of the N ras gene. The ratios between diagnosis mitochondrial genomes/cell and remission mitochondrial genomes/cell were calculated.

Results

DGGE was performed on diagnosis DNA in 21 patients with AML and 16 patients with ALL. For these 37 patients there was excellent concordance between sequencing and DGGE. For these 37 patients, 145 segments had been studied by DGGE. The results are shown in Table 3 and they indicate that homogeneity as evidenced by DGGE had a sensitivity of 93% and a specificity of 98% for detection of mutations

On DGGE, the mutated band(s) of the leukaemic clone present at diagnosis were also frequently visible in the remission marrow, being observed in 5 of the 6 AML patients and 3 of the 9 ALL patients in whom DGGE was performed and in whom a leukaemic band was present at diagnosis. The band at remission was usually faint but it was quite strong in 1 patient. This patient had acute promyelocytic leukaemia. Review of the cytogenetics revealed that the majority of metaphases at morphological remission were still leukaemic and confirmed that the DGGE finding indicated the presence of leukaemia rather than homeoplasmy.

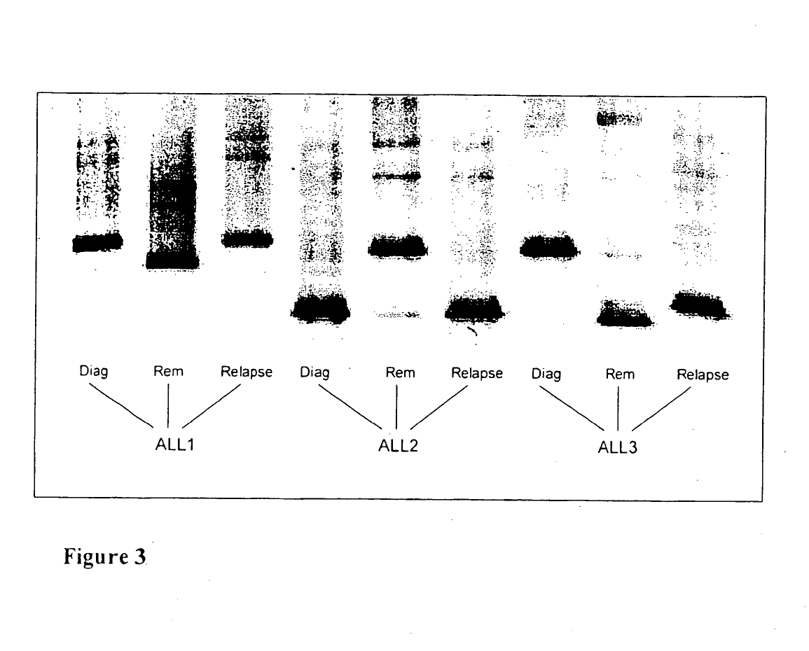

Of the 15 ALL patients for whom relapse DNA was also studied, one or more homogeneous DGGE bands were observed in 8. Of these 8 patients, the same DGGE band(s) and sequence were present at relapse in 4. In the other 4 patients, DGGE bands and sequences differed from those present at diagnosis. In 1 patient the DGGE band which had been observed in the diagnosis DNA was no longer observed in the relapse DNA. In the other 3 patients one or more new DGGE bands were observed. In 2 of these 3 patients DGGE of the remission sample showed that the relapse band could also be detected. The DGGE observations in these 4 patients suggest a process of clonal evolution which led to two principal clones being present at diagnosis, one of which predominated but which was relatively sensitive to chemotherapy, the other of which was smaller in size but resistant to chemotherapy and responsible for relapse. In FIG. 3 are shown DGGE results illustrating some of the varied findings described above.

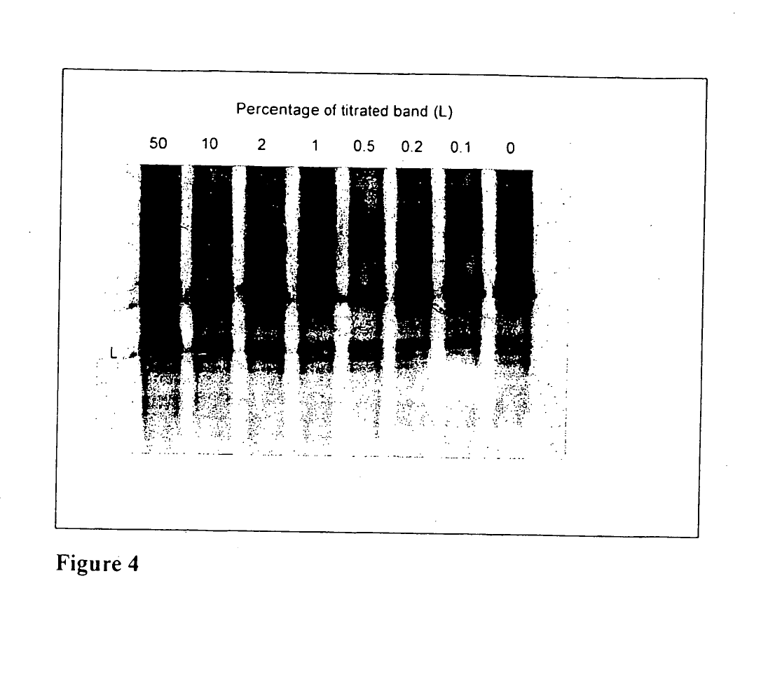

Results from 1 of the 2 experiments designed to determine the level of detection achievable by DGGE are shown in FIG. 4. In both experiments, the level of detection achieved was approximately 1% in terms of the mass of “leukaemic” mitochondrial DNA mixed with “non-leukaemic” mitochondrial DNA. The ratio between number of mitochondria/cell at diagnosis and remission was 2.4×/÷1.8 (mean×/÷1 SE) for 5 patients with AML and 4.3×÷1.8 (mean×/÷1 SE) for 6 patients with ALL. Taken together, the results suggest that the level of detection of a minor leukaemic cell population would be 0.2-0.5%.

The fact that in these preliminary studies a sensitivity of 0.2-0.5% was achievable by DGGE and that in many patients in morphological remission the leukaemic band could still be seen in the remission marrow, indicates that mitochondrial mutations are candidate molecular markers for monitoring of MRD in leukaemia. A theoretical limitation of the use of DGGE for monitoring MRD is that clonal evolution might result in a relapse clone having a different mitochondrial sequence to that present in the diagnosis clone. However the ability of DGGE to detect mutations at many locations in the segment being studied makes it unlikely that a clone bearing a new but still mutated sequence would be missed.

TABLE 3
Comparison of detection of mutations by sequencing
or homogeneity by DGGE, either in patients overall
or in individual DNA segments.

| (a) patients | | | | (b) DNA segments | | | |
|---|---|---|---|---|---|---|---|
| | | Sequencing | | | | Sequencing | |
| | | + | − | | | + | − |
| DGGE | + | 17 | 0 | DGGE | + | 50 | 2 |
| | − | 1 | 19 | | − | 4 | 89 |

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

1. A method of detecting a clonal population of cells in a biological sample, which clonal cells are characterised by a diagnostically distinctive nucleic acid region, said method comprising co-localising the subject nucleic acid regions derived from said sample, which co-localisation is based on nucleotide sequence identity, and qualitatively and/or quantitatively detecting the levels of said co-localised nucleic acid regions wherein a higher level of a co-localised nucleic acid region population relative to background levels is indicative of the presence of a clonal population of cells in said sample.

2. A method for diagnosing and/or monitoring a clonal population of cells in a mammal, which clonal cells are characterised by a diagnostically distinctive nucleic acid region, said method comprising co-localising the subject nucleic acid regions derived from a biological sample derived from said mammal, which co-localisation is based on nucleotide sequence identity, and qualitatively and/or quantitatively detecting the levels of said co-localised nucleic acid regions wherein a higher level of a co-localised nucleic acid region population relative to background levels is indicative of the presence of a clonal population of cells in said sample.

3. The method according to claim 1 or 2 wherein said clonal population of cells is a neoplastic clonal population.

4. The method according to claim 3 wherein said neoplastic population of cells corresponds to a leukaemia, lymphoma or myeloma.

5. The method according to claim 4 wherein said leukaemia is actue myeloid leukaemia or acute lymphoblastic leukaemia.

6. The method according to claim 1 or 2 wherein said clonal population of cells is a non-neoplastic clonal population of cells.

7. The method according to claim 6 wherein said non-neoplastic population of cells corresponds to a myelodysplasia, polycythaemia vera or a myeloproliferative syndrome.

8. The method according to claim 3 or 6 wherein said clonal population of cells is a clonal immune cell population.

9. The method according to claim 8 wherein said immune cell is a T cell or a B cell.

10. The method according to claim 1 or 2 wherein said clonal population of cells is a clonal microorganism population.

11. The method according to any one of claims 1-10 wherein said nucleic acid region is a DNA region.

12. The method according to claim 11 wherein said diagnostically distinctive DNA region is mitochondrial DNA or a microsatellite.

13. The method according to claim 12, wherein said mitochondrial DNA is mitochondrial D loop DNA.

14. The method according to claim 5 wherein said nucleic acid region is a DNA region and said diagnostically distinctive DNA region is mitochondrial D loop DNA.

15. The method according to any one of claims 1-14 wherein said co-localisation is achieved utilising any one of the techniques of:
(i) Denaturing gradient electrophoresis.
(ii) Temperature gradient denaturing electrophoresis
(iii) Constant denaturing electrophoresis
(iv) Single strand conformational electrophoresis
(v) Denaturing high performance liquid chromatography
(vi) Microassays
(vii) Mass spectrometry

16. The method according to claim 14 wherein said co-localisation is achieved utilising denaturing gel or capillary electrophoresis.

17. A method for diagnosing and/or monitoring a mammalian disease condition characterised by the presence of a clonal population of cells, which clonal cells are characterised by a diagnostically distinctive nucleic acid region, said method comprising co-localising the subject nucleic acid regions derived from a biological sample derived from said mammal, which co-localisation is based on nucleotide sequence identity and qualitatively and/or quantitatively detecting the levels of said co-localised nucleic acid regions wherein a higher level of the co-localised nucleic acid region population relative to background levels is indicative of the presence of a clonal population of cells in said sample.

18. The method according to claim 17 wherein said clonal population of cells is a neoplastic clonal population.

19. The method according to claim 18 wherein said disease condition is leukaemia, lymphoma or myeloma.

20. The method according to claim 19 wherein said leukaemia is actue myeloid leukaemia or acute lymphoblastic leukaemia.

21. The method according to claim 17 wherein said clonal population of cells is a non-neoplastic clonal population of cells.

22. The method according to claim 21 wherein said disease condition is myelodysplasia, polycythaemia vera or a myeloproliferative syndrome.

23. The method according to claim 18 or 21 wherein said clonal population of cells is a clonal immune cell population.

24. The method according to claim 23 wherein said immune cell is a T cell or a B cell.

25. The method according to claim 17 wherein said clonal population of cells is a clonal microorganism population.

26. The method according to any one of claims 17-25 wherein said nucleic acid region is a DNA region.

27. The method according to claim 26 wherein said diagnostically distinctive DNA region is mitochondrial DNA or a microsatellite.

28. The method according to claim 27, wherein said mitochondrial DNA is mitochondrial D loop DNA.

29. The method according to claim 20 wherein said nucleic acid region is a DNA region and said diagnostically distinctive DNA region is mitochondrial D loop DNA.

30. The method according to any one of claims 17-29 wherein said co-localisation is achieved utilising any one of the techniques of:
(i) Denaturing gradient electrophoresis.
(ii) Temperature gradient denaturing electrophoresis
(iii) Constant denaturing electrophoresis
(iv) Single strand conformational electrophoresis
(v) Denaturing high performance liquid chromatography
(vi) Microassays
(vii) Mass spectrometry

31. The method according to claim 30 wherein said co-localisation is achieved utilising denaturing gel or capillary electrophoresis.
